(19) United States
(12) Patent Application Publication
(10) Pub. No.: US 2005/0020962 A1
(43) Pub. Date: Jan. 27, 2005

(54) Diagnostic algorithms for a csf physiologic controller

(76) Inventors: Sanford Reich, Providence, RI (US); James E. Sluetz, N. Attleboro, MA (US)

Correspondence Address: NIELDS & LEMACK, 176 EAST MAIN STREET, SUITE 7, WESTBORO, MA, 01581, US

(21) Appl. No.: 10/500,391

(22) PCT Filed: Jan. 2, 2003
(86) PCT No.: PCT/US03/00095

Related U.S. Application Data
(60) Provisional application No. 60/345,089, filed on Jan. 4, 2002.

(51) Int. Cl. A61M 5/00; G01M 3/02

(52) U.S. Cl. 604/8; 73/37

(57) ABSTRACT

The Cerebrospinal Fluid (CSF) Physiologic Controller is an implantable active battery-operated device that is microprocessor controlled via algorithms stored in its memory. The controller also contains numerous diagnostic features, which enable the physician to monitor the operation of the system, as well as several key patient parameters non-invasively, by performing a set of algorithms.

This application claims priority of provisional application Ser. No. 60/345,089 filed Jan. 4, 2002, the disclosure of which is hereby incorporated by reference.

### BACKGROUND OF THE INVENTION

The human skull is primarily occupied by brain tissue and the supporting blood vessels. About ten percent of this volume is clear fluid with small amounts of dissolved protein, sugar and salts. This fluid is known as cerebrospinal fluid (CSF). This CSF fluid cushions the delicate brain and spinal cord tissues from injuries and maintains the proper balance of nutrients and salts around the central nervous system.

A system of four interconnecting cavities, known as ventricles, in the brain provide pathways through which the CSF circulates from deep within the brain, around the spinal column, and over the surfaces of the brain. CSF is continually being created. In fact, about three to five times the volume contained in the skull at any point in time is produced on a daily basis

Hydrocephalus is an abnormal accumulation of CSF in the ventricles. Hydrocephalus can be present at birth (congenital), acquired as a result of brain trauma, or can occur in adults in a condition known as normal pressure hydrocephalus.

Normally, almost all of the CSF is absorbed into the bloodstream, thus maintaining the delicate balance between CSF production and absorption. This fluid system becomes unbalanced when the rate of CSF production in the ventricles is greater than the rate of CSF absorption into the bloodstream. The excess fluid causes increased intraventricular pressure (IVP). A high level of pressure for any sustained period can lead to serious complications.

The most common treatment for hydrocephalus is shunt therapy; a surgical procedure in which a hydrocephalus valve system is usually implanted while the patient is under general anesthesia. In this commonly used procedure, a small hole is made in the skull and the protective membrane overlaying the brain. An incision is made in the abdomen and the valve unit and associated tubing are introduced under the skin between the scalp and the abdominal incisions. Usually one ventricular cannula is inserted into the lateral ventricle and connected to the drainage tube, which is inserted in the abdominal cavity. The drainage cannula may also be introduced through a neck incision and passed through various blood vessels until the tip of the cannula is positioned in the right atrium of the heart. This system is intended to allow CSF from the ventricle to travel through the implanted tubes into either the abdominal cavity or the heart, where it is then absorbed into the bloodstream.

Under-drainage, in which the fluid is not removed quickly enough, is a common problem of the shunt system. Sometimes under-drainage may be due to the shunt cannula breakage or disconnection. Valve blockage is relatively uncommon. This breakage or disconnection disrupts the new path made for the CSF and causes increased pressure in the ventricles. Rapid increase in IVP may result in loss of consciousness, and emergency treatment is required. However, in most cases, the onset is more gradual, and can follow a minor illness, such as a cold. Headaches increase in frequency and severity, often worse upon waking in the morning. Vomiting and dizziness may also occur, and sometimes there may be other symptoms, which vary from patient to patient.

Over-drainage, in which the shunt allows CSF to drain from the ventricles more quickly than it is being produced, is also a common problem in shunt therapy. If this happens suddenly, such as soon after the shunt is inserted, then the ventricles of the brain may collapse, tearing delicate blood vessels on the outside of the brain and causing a hemorrhage. This can be trivial or it can cause symptoms similar to those of a stroke. The blood may have to be removed, and in some cases, if this is not done, it may be the cause of epilepsy later. If the over-drainage is more gradual, the ventricles collapse gradually to become slit-like. This often interferes with the function of the shunt, causing the opposite problem, high IVP. Unfortunately, the slit ventricles may not always increase is size, resulting in headache and vomiting.

The symptoms of over-drainage can be very similar to those of under-drainage with an important difference. With over-drainage, headaches often become worse getting up from a supine (horizontal) position. This is because the change in position causes excessive drainage to occur, since gravity forces more CSF to drain. With under-drainage, headaches caused by high IVP often become worse on waking in a supine position. This is because little CSF is drained in the horizontal position, causing an increase in IVP. The best way to distinguish between these two conditions is to monitor the IVP over 24 hour periods.

The drainage rate of the shunts varies depending on the patient's relative position. In an upright position, an increased rate of CSF flow is generated, since gravity serves to create siphoning pressure, which will aid in the drainage process. In the supine, or horizontal, position, drainage is caused solely by the imbalance of pressure. Current shunt therapy devices are not designed to effectively treat over-drainage. These devices still maintain a large negative IVP (over-drainage) when the patient is in the upright position. A change of valve to a higher pressure cannot be relied upon to cure it, though it appears to do so in some cases. Anti-siphon devices, which consist of a small button inserted into the shunt tubing, may sometimes solve the problem. Some shunts have these built-in, but neurological opinion varies as to whether they should be used. To change a valve pressure, surgery is necessary to remove the valve and insert another. A relatively new shunt, the ‘programmable’ or adjustable shunt, is intended to allow adjustment of the working pressure of the valve without surgery. This valve contains magnets that allow the valve pressure setting to be altered by a transcutaneous magnetic field placed over the scalp. This is useful where the need for a valve of a different pressure arises, but the adjustable valve is no less prone to the over-drainage issue than any other and it cannot be used to treat this condition.

Normal pressure hydrocephalus (NPH) is an accumulation of cerebrospinal fluid that causes the ventricles to become enlarged with a return to normal pressure. The name of this condition is misleading, however, because some patients have fluctuations of IVP from high to normal to low. In most cases of NPH, it is not clear what causes the CSF pathways to become blocked.

Normal intraventricular pressure (IVP) is between 10-15 mm Hg in the supine position and −5 mm Hg in the upright position.

Adult-onset normal pressure hydrocephalus described those cases that occur in older adults (age 50 and older). The majority of the NPH population is 60 years or older. In the majority of cases of NPH, the cause is unknown. In some cases, NPH can develop as a result of a head injury, cranial surgery, subarachnoid hemorrhage, meningitis, tumor or cysts, as well as subdural hematomas, bleeding during surgery and other infections. The syndrome of NPH is usually characterized by complaints of gait disturbance (difficulty walking), mild dementia and impaired bladder control.

Hydrocephalus is often classified as either communicating or non-communicating. In the former, the problem is usually failure to absorb the CSF at the end of the system, whereas in the latter, there is blockage of the CSF pathways within the ventricular system.

In summary, the limitations of the current implantable shunt technologies are as follows. The current shunt systems use passive components such as check valves to regulate the flow of CSF. These passive check valves are designed to open when a predetermined pressure drop exists across the check valve. Short-term changes, such as when the patient rises from a horizontal position to a standing position, may cause excess drainage because of the added siphoning of the vertical tubing. In the longer term, passive check valves are not able to automatically maintain normal IVP by adjusting CSF drainage if the patient experiences changes in CSF generation. Note that the selection of a pressure valve may result in a compromise between under-drainage in the supine position and over-drainage in the upright position.

The current shunt systems have no method for non-invasively measuring the CSF drained flow rate. Therefore, once installed, it is difficult to monitor the shunt's operation.

The current shunt systems cannot monitor IVP except during an invasive procedure, requiring a needle. Sustained low or high IVP may lead to serious complications.

The current shunt systems do not have the capability to monitor, store, and transmit data related to CSF flow, IVP or cannula operation.

The current invention is primarily targeted toward the adult-onset normal pressure hydrocephalus population. It is the objective of the present invention to create a set of algorithms that enable the physician to overcome all of the shortcomings listed above using non-invasive techniques.

### SUMMARY OF THE INVENTION

The limitations of the current shunt therapy for hydrocephalus have been overcome by the present invention. The CSF Physiologic Controller is an implantable active battery-operated device that is microprocessor controlled via algorithms stored in its memory. It is a multi mode drainage system that contains at least two flow paths: a low resistance flow path for when the patient is in the supine or substantially supine position and a flow path containing a programmable variable check valve to prevent over-drainage when the patient is in the upright or substantially upright position. The Controller also contains numerous diagnostic features, which enable the physician to monitor the operation of the system, as well as several key patient parameters non-invasively

### BRIEF DESCRIPTION OF THE DRAWINGS

FIG. 1 is a flow schematic of the CSF Controller;

FIG. 2 is a view of the variable check valve, located within the CSF Controller;

FIG. 3 is a graph demonstrating the check valve's performance over a range of conditions;

FIG. 4 is the preferred implantation of the CSF system in the patient; and

FIG. 5 is a detailed view of the CSF Controller.

### DETAILED DESCRIPTION OF THE INVENTION

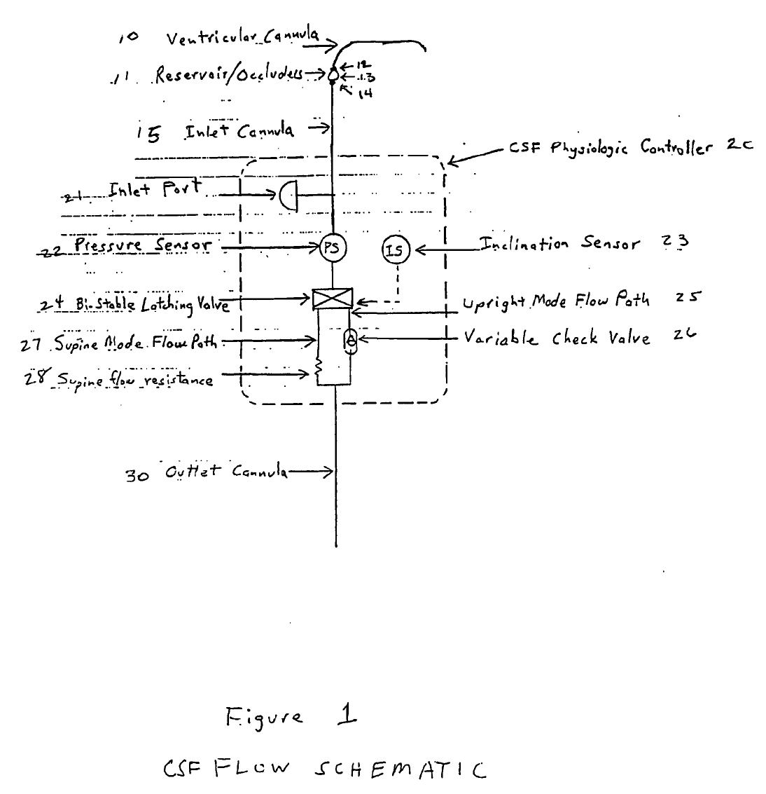

The CSF Physiologic Controller is a multi mode drainage system that contains at least two flow paths: (1) a supine mode: a low resistance flow path for when the patient is in the supine or substantially supine position and (2) an upright mode: a flow path containing a programmable variable check valve to prevent over-drainage when the patient is in the upright or substantially upright position. A bi-stable latching valve directs the CSF flow to either the low resistance path or the check valve path based on an inclination sensor within the CSF Physiologic Controller. If the inclination sensor angle is below a programmable critical angle, the bi-stable latching valve directs flow to the low resistance path. If the inclination sensor angle is equal to or above a critical programmable angle, the bi-stable latching valve directs flow to the check valve path. For purposes of illustration, a dual mode controller will be described; however, the present invention is not limited to only two modes. FIG. 1 shows the flow schematic of the CSF Controller System.

The ventricular cannula 10 is typically implanted in the ventricle of the patient's brain. It serves as the source for the CSF fluid into the system. The ventricular cannula is in fluid communication with the reservoir/occluders device 11. This device is implanted just beneath the scalp and can be actuated by pressing on the scalp. This device contains a reservoir 13 for holding CSF fluid. On either side of the reservoir is a manual blocking mechanism, known as an occluder. The one nearer to the ventricle is known as the proximal occluder 12, while the other is the distal occluder 14. These occluders allow the physician to interrupt the flow of CSF to perform a number of in-office non-invasive diagnostics.

The distal occluder 14 is in fluid communication with the inlet cannula 15, which is a tube that is in fluid communication with the CSF Physiologic Controller 20. The Physiologic Controller is preferably located below the clavicle in the pectoral area. It regulates the flow of CSF through it, and the outgoing CSF flows into the outlet cannula 30. This outlet cannula is implanted such that its distal (far) end is inserted into the peritoneal space or inserted intravenously with its distal tip in the right atrium of the heart.

FIG. 4 shows the preferred implantation site for the CSF controller. The ventricular cannula is inserted into the ventricle of the brain with its distal end in fluid communication with the reservoir/occluder 11, which is placed just under the scalp. The inlet cannula 15 traverses the body from the scalp to the CSF Controller 20, which is located in the pectoral area of the chest. The outlet cannula 30 has its distal end placed in either the abdominal cavity or the right atrium of the heart.

Referring back to FIG. 1, the CSF Physiologic Controller 20 contains all of the mechanisms required to implement the CSF flow. The inlet port 21 is used to access fluid in the inlet cannula. The side branch, that is only used occasionally, is not part of the main fluid path so as to prevent accumulation of protein particle with the side branch. The inlet port 21 is preferably a silicone rubber septum assembly through which a doctor may insert a needle for the purpose of sampling the CSF, to inject a dye for diagnostic purposes, or inject saline to check for flow blockages. In this implementation, the port is also used to periodically calibrate a pressure sensor.

The inlet cannula flows into the pressure sensor component 22, located within the CSF controller 20. The purpose of this sensor is to determine the relative pressure of CSF at the inlet cannula. The following is for illustrative purposes only; a number of different embodiments could be used to implement the pressure sensor. In this embodiment, the pressure sensor component is actually two distinct MEMS (Micro-Electro-Mechanical Systems) absolute pressure sensing silicon elements. The two MEMS silicon pressure-sensing elements may be attached to a common vacuum. The non-vacuum sides of each are oil-coupled to the force-collecting diaphragms. The top force-collecting diaphragm is integral with a flat portion of the CSF fluid path and measures the absolute pressure in the CSF path. The lower force-collecting diaphragm is in communication with the outside bottom portion of the device and measures the absolute pressure on the outside of the device. This outside pressure sensing element measures the tissue pressure of the implanted device and closely tracks the atmospheric pressure. A mechanical guard over the outside force-collecting diaphragm protects it from mechanical forces that may produce pressure artifacts. The difference between the two absolute pressure sensors is the gauge pressure of the CSF at the inlet to the Controller. This pressure is indicative of the intraventricular pressure (IVP). In the supine position, this reading is roughly equivalent to the IVP. In the upright position, this reading is the IVP plus the siphon pressure created by the shunt. By using the inclination sensor, it is possible to determine the actual IVP of the patient regardless of the inclination angle. In normal operation, the pressure sensor monitors the intraventricular pressure (IVP) not continuously, but periodically, for example, every 2-5 minutes. These readings can be stored in the Controller's memory. Using the telemetry capability of the Controller to download the information to the external programmer, the physician may review daily changes in IVP to diagnostic purposes. For example, the physician may choose to do this when a patient complains of headaches. The CSF Controller can sample the pressure sensor at any time to determine the IVP, as measured at the input to the Controller.

The inclination sensor 23 is a gravity-detecting sensor that is used to determine the patient's inclination angle. It is used to control the multi mode CSF Physiologic Controller. This sensor also detects patient activity, such as when the patient is resting or moving about. Both the inclination and activity functions may be utilized to control the bi-stable latching valve 24.

The bi-stable latching valve 24 directs the CSF flow to the low resistance, supine mode path 27 when the inclination sensor 23 indicates that the patient is in a supine or substantially supine position; or the upright mode flow path 25 when the inclination sensor indicates that the patient is in an upright position.

The supine mode flow path 27 includes a supine flow resistance 28, which is designed to prevent against under-drainage and keep the IVP within the normal upper limit of 15 mm Hg. In this embodiment, the supine flow resistance is simply the resistance of the cannula in the supine mode flow path.

The upright mode flow path 25 provides a variable high resistance flow path that is designed to prevent over-drainage. The variable high resistance flow path is provided by a variable check valve 26 whose cracking pressure is automatically adjusted based on the inclination angle.

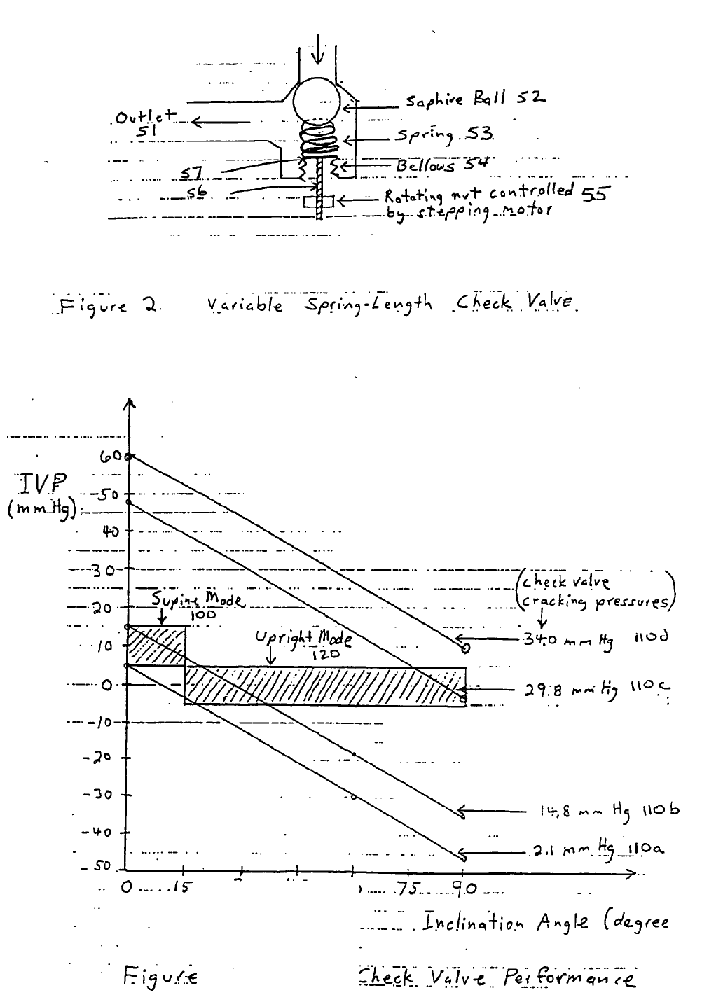

FIG. 2 shows a suitable design for a variable check valve. This diagram is for illustrative purposes only, and the check valve is not limited to a particular valve embodiment. The CSF flow originates at the inlet 50. A ball 52 serves to block the CSF from passing from the inlet 50 to the outlet 51. The ball 52 is preferably constructed of a material not deleterious to the application, such as sapphire, which does not interact with the cerebrospinal fluid. The ball is preferably small in diameter in order to ensure the best seal when the ball is resting on the inlet 50. For CSF to pass to the outlet, the pressure of the CSF at the inlet 50 must exceed the pressure exerted by the spring 53. The point at which this occurs is known as the cracking pressure. At this point, the ball will rise and allow the CSF to flow through the inlet 50 and onto the outlet. The spring 53 is located between the sapphire ball 52 and a horizontal platform 57. This horizontal platform can be moved both up and down by rotating screw 56. As the horizontal platform is moved up, the cracking force increases. Likewise, as the horizontal platform is lowered, the cracking force decreases. Bellows 54 covers the horizontal platform to insure that the valve is fluid-tight. The rotating screw 56 is controlled by a nut 55, which in turn is controlled by a stepping motor (not shown). The stepping motor is controlled by the microprocessor. In this embodiment, the stepping motor controls the nut as a function of the patient's inclination angle, as determined by the inclination sensor 23.

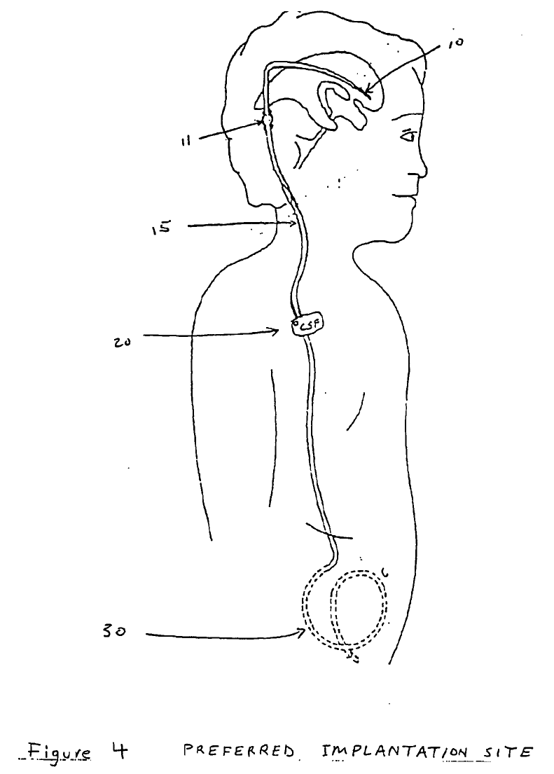

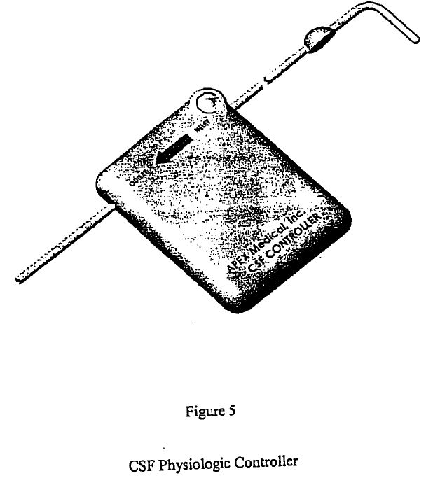

FIG. 3 graphically illustrates the operation of the check valve. This particular graph is done for purposes of illustration, and the invention is not limited to this functionality. This graph shows intraventricular pressure graphed as the vertical axis, with patient's inclination angle as the horizontal axis. For clarity, 0 degrees denotes a person in the completely horizontal position, while 90 degrees is a patient in the fully upright position. In this example, the siphon length was 62 cm. Four diagonal lines 110a-d show lines of constant check valve cracking pressure. As an example, if the check valve cracking pressure were held constant at 2.1 mm Hg, as in line 110a, the IVP would be 5 mm Hg in the supine position. The IVP would decrease as the inclination angle increased, reaching a value of about −45 mm Hg when the patient is fully upright. Similarly, line 110d illustrates that for a cracking pressure of 34.0 mm Hg, the IVP is 60 mm Hg when the patient is fully supine and about 10 mm Hg when the patient is completely upright. Lines 110b and 110c show similar trends at 14.8 mm and 29.8 mm, respectively. The shaded area, supine mode 100, denotes the desired IVP when the patient is in the supine or substantially supine position. As used herein, substantially supine is defined as less than about 15 degrees of inclination (accordingly, substantially upright is an inclination angle greater than about 15°). In the present invention, this result is achieved using the low resistance supine mode flow path 27 in the CSF. Once the patient's inclination angle exceeds about 15 degrees, the CSF uses the upright mode flow path 25. In this mode, the desired IVP range is shown in the shaded area, upright mode 120. At 15 degrees, the valve cracking pressure is between 2.1 mm and 14.8 mm in order to achieve an IVP of −5 to 5 mm Hg. As the patient becomes more upright (i.e., the inclination angle approaches 90°), the cracking pressure increases in order to maintain the desired IVP. When the patient is fully upright, the cracking pressure is about 29.8 mm Hg in order to maintain the proper IVP.

In addition to the elements described above, which are part of the flow paths, there is a microprocessor-based subsystem internal to the CSF Controller. This subsystem preferably comprises a microprocessor, its associated memory, a Real Time Clock, a wireless transceiver, a piezo electric buzzer and other essential electronics. An internal battery powers this subsystem. The microprocessor is responsible for monitoring and controlling many of the operations enumerated above, such as monitoring the inclination sensor, adjusting the check valve cracking pressure, and monitoring the pressure sensor. The microprocessor is also capable of receiving commands and returning status to the external programmer via the wireless transceiver. The memory is used to store data requested by the external programmer, such as pressure readings, inclination angle, and time. This data can be transmitted back to the external programmer as requested. The Real Time Clock is used to enable the Controller to perform certain diagnostics at specific times. The piezo electric buzzer is used to alert the patient of certain conditions. As an example, the buzzer may sound once per hour to indicate a low battery condition. This method of warning is used currently by those skilled in the art for a variety of device, such as pacemakers.

In conjunction with the CSF Controller, there is an accompanying external programmer. This programmer is preferably used by a physician, and is used to program critical parameters in the CSF Controller, retrieve stored information from the CSF Controller, and perform other types of communication with the CSF Controller. The external programmer can also be used Lo perform a number of diagnostic procedures in conjunction with the CSF Controller. The external programmer permits the physician to program the desired critical angle at which the CSF Controller switches from supine to upright mode. The programmer can also be used to program the valve cracking pressures as a function of the patient inclination angle. This profile is used to create the patient unique version of FIG. 3. The programmer also contains a MEMS based barometer that is used to calibrate the tissue pressure sensor in the CSF Controller.

The programmer can take many different physical forms. It preferably comprises the following set of components:
1. a processor unit to perform the necessary algorithms and calculations;
2. an internal memory to store data received from the controller, and other relevant information;
3. a data input device to accept input from the physician;
4. a data output device to display data to the physician;
5. and a communication port to transmit information to the CSF Controller.

The programmer, which is preferably handheld, can be a custom developed apparatus, or can be an existing device, such as a Palm™ handheld or laptop computer. In the scenario where a Palm™ handheld is used, the criteria above are met as follows. The processor unit and internal memory are standard elements of the Palm™ handheld. The data input device is the touch screen of the device, or the optional keyboard. The data output device is also the touch screen. Lastly, the communication to the CSF Controller is performed by an optional wireless module that can be connected to the Palm™ handheld.

The current invention has the ability to allow non-invasive measurements of key parameters, regarding correct shunt operation. By performing several algorithms, the physician is able to determine conditions, such as blocked cannulas, cranial compliance, and CSF flow rate. Currently, in CSF shunt therapy, there is no easy, cost effective method of determining these parameters. The following parameter definitions are used in these algorithms:

R_{D}=shunt flow resistance distal to the pressure sensor (from the CSF Controller to the distal end of the shunt), measured in (mm Hg)/(ml/hour)

Rₚ=shunt flow resistance proximal to the pressure sensor (from the ventricle to the CSF Controller), measured in (mm Hg)/(ml/hour)

C=Cranial compliance, measured in ml/(mm Hg)

IVP(t)=intraventricular pressure as a function of time (t).

P(t)=CSF gauge pressure (mm Hg) measured in the device as a function of time (t).

Φ=patient inclination angle, measured in degrees.

PH(Φ)=pressure height correction, between the head and the CSF Controller as a function of Φ.

PS(Φ)=siphon pressure for the entire shunt as a function of Φ.

PD=pressure at the distal end of the shunt, measured in mm Hg.

T_{1/2}=time required for the pressure to decay to one half of the total pressure decay.

F=calculated supine shunt flow rate (ml/hour)

F(t)=calculated supine flow rate as a function of time. □t=the time between successive sampled values

The following algorithms allow a physician to non-invasively monitor the operation of the CSF shunt, as well as the health of the patient. This information was previous only available through invasive techniques, if at all. The use of these algorithms allows for easy monitoring and provides a proactive method of patient management.

The first algorithm, Algorithm 1, is used to compute R_{D}. R_{D} represents the distal flow resistance of the shunt in the supine mode from the pressure sensor to the distal end of the shunt (in either the peritoneal space or the venous return in the right atrium). The principle behind this algorithm is that the exponential pressure decay time for a given pressure to reach the PD pressure when the shunt is temporarily occluded at the distal end of the reservoir is proportional to the resistance. At the time of shunt manufacture, this relationship between decay time and flow resistance is calibrated. Measurements of the transient pressure decay times for applied initial condition pressures and a steady-state flow pressure versus flow measurement for the distal cannula determine the values for T_{1/2}, and R respectively. Using these, a value for the calibration constant, Kₘ can be calculated. Note that trimming of the distal shunt length during surgical implantation may require a correction factor. This distal flow is important because it primarily monitors the distal end of the shunt, where blockages are likely to occur. If these can be detected non-invasively, it allows the physician to quickly understand if the shunt is working properly. This measurement can be performed in a convenient location, such as at a physician's office using the external programmer with the patient lying in the supine position.
1. Calibrate the tissue reference sensor in the Controller with the barometric sensor in the external programmer. This insures that pressure is measured in an identical fashion.
2. Have the patient lie motionless in a horizontal position. This causes the CSF Controller to operate in the supine flow mode.
3. Measure and record the pressure sensor's reading, which is the IVP and transmit the data to the external programmer.
4. Program the Controller to have the pressure sensor to measure and record at a high sample rate (approximately 5-10 samples per second for about 10 seconds).
5. Manually occlude the distal end of the reservoir for the same time period. This effectively blocks any newly formed CSF from entering the inlet cannula. Instead, it is stored in the reservoir 13 for the duration of the test.
6. Use the programmer to download the pressure time profile data for this time period from the Controller to the programmer.
7. Measure and record the pressure at the start of the exponential decay (which is equal to IVP) and the stable pressure at the end of the exponential decay (PD).
8. Measure the time required for the pressure to decay to one half of the total pressure decay. This time is called T_{1/2}.
9. Knowing that resistance is proportional to the exponential decay time, use the following formula to calculate R_{D}: R_{D}=T_{1/2×K}ₘ, where Kₘ is the manufacture calibration constant.

The second algorithm, Algorithm 2, is used to compute F. F is the supine flow rate that the physician may calculate using the data obtained from the first algorithm. Flow rate is important to know because it verifies that the shunt is working correctly. For example, if there is an upstream blockage (for example, in the inlet cannula), the pressure sensor provides an erroneous measurement of IVP because it is unaware of the blockage. This will result in an erroneous flow calculation. Measuring a shunted flow that is within an expected range provides a reality check that the active shunt device is not isolated from the brain fluid (CSF).

Using the principle that flow through a fluid resistance may be calculated by measuring the pressure difference across the fluid resistance, the flow is obtained by:

F=(IVP−PD)/R_{D} where IVP, PD, and RD were all obtained by performing Algorithm 1.

The third algorithm, Algorithm 3, is used to compute compliance (C). The definition of compliance is the change in the volume of CSF divided by the change in IVP pressure. Compliance in the brain, C, is a clinical measure of the patient's brain state. A low compliance means that continued high pressure in the brain has enlarged the ventricles and compressed the brain matter. This condition may be verified with MRI, but the compliance measurement is simpler and more cost effective. This can be performed at a convenient location, such as a physician's office, using the external programmer with the patient in an upright position. When the patient is in this position, the CSF maintains the IVP at approximately −5 mm Hg. At this time, the external programmer is used to temporarily switch the CSF Controller to operate in supine mode, and the pressure sensor records the IVP as it decays exponentially. The IVP at the start and the end of the exponential decay are recorded and the flow rate is computed for each pressure reading in the pressure decay. The total volume of CSF fluid shunted during the pressure decay is determined by integrating the flow rate during the pressure decay time period. The following steps outline the procedure:
1. Have the patient sit motionless in an upright position.
2. Use the external programmer to have the CSF Controller measure and record the pressure sensor reading, which is the initial IVP.
3. Use the external programmer to switch the CSF Controller to operate in supine mode.
4. Use the external programmer to transmit the pressure data time profile stored in the Controller to the external programmer.
5. Compute the instantaneous flow rate for each sample pressure time. Knowing that flow is pressure divided by resistance, use the following formula:
   F(t)=(1/R_{D})×[IVP(t)+PS(Φ)−PD]
for IVP(0)≦IVP(t)≦IVP(k□t) for each of k sample pressure times.
RD and PD were computed in Algorithm 1.
PS(Φ) is the siphon pressure for the entire shunt system. It can be calculated by multiplying the shunt tube length by a conversion constant (50.7 mm Hg/27.7 inches). This resultant product is then multiplied by cosine(Φ) to yield the siphon pressure.
6. Integrate the instantaneous flow rates for each sample pressure time. This will yield the total flow of CSF fluid.
   □V=Σ[F(t)×□t] for 0≦t≦k□t
7. Cranial compliance is defined as the amount of change in CSF volume, divided by the change in IVP pressure.
   C=□V/[IVP[0]−IVP(k□t)], in ml/(mm Hg)

The fourth algorithm, Algorithm 4, is used to calculate the proximal shunt flow resistance (Rₚ), or the resistance from the ventricle to the CSF Controller. A qualitative method for determining if the ventricular cannula is plugged at the tip is to use the reservoir 12, together with the distal occluder 13 to manually flush a small controlled volume of CSF fluid into the ventricle. This method, which is currently known by those skilled in the art, will detect a blocked cannula tip but is not sensitive enough to monitor a trend or predict future occlusions. A quantitative method is to measure the decay time for the entire cannula system using the cranium compliance that was calculated in Algorithm 3. An estimation of the exponential decay time of the entire system can be expressed as:
T_{1/2}=0.7×(R_{D}+Rₚ)×C
Where R_{D} and T_{1/2} were calculated in Algorithm 1
C was calculated in Algorithm 3.

Therefore, the proximal resistance can be determined by:
Rₚ=T_{1/2}/(0.7×C)−R_{D}

The final algorithm, Algorithm 5, is used to automatically monitor the trend in the shunt flow resistance on a nightly basis in a way that is transparent to the patient. This trend monitoring will alert the patient, preferably by sounding the implanted piezo electric buzzer, to visit the physician before a crisis occurs. This minimizes therapy downtime and allows the patient and physician to be proactive. This algorithm is used while the patient is in the supine position, as monitored by the inclination sensor. Using the Real Time Clock in the CSF Controller, this algorithm can be programmed to be performed in the middle of the patient's sleep cycle, such as in the middle of the night.
1. Program the CSF Controller using the external programmer to execute Algorithm 5 in the middle of the patient's typical sleep period.
2. At the programmed time, the Controller confirms that the patient is indeed in the supine position by monitoring the inclination sensor.
3. The CSF Controller records the reading of the pressure sensor, which measures the IVP.
4. The CSF Controller switches to upright mode with the check valve set to a cracking pressure that would require about 20 mm Hg to open.
5. The CSF Controller monitors the IVP until it rises to a programmable value (approximately 2-6 mm Hg) above the IVP measured in step 3.
6. The CSF Controller switches back to supine mode.
7. The CSF Controller measures the pressure decay time (T_{1/2}) required for the IVP to drop to half the difference programmed in step 5.
8. The CSF Controller stores this time in its memory.
9. The CSF Controller alerts the patient, using the implanted piezo electric buzzer, if a significant change in T_{1/2}.

The pressure decay time is proportional to the total shunt resistance in the supine mode. Thus, any significant change in the pressure decay time may be a predictive warning of a future shunt occlusion. The alerted patient should contact the physician for further diagnostic evaluation.

1. A system for non-invasively monitoring the operation and performance of an implanted cerebrospinal shunting system, comprising an implanted controller; said controller further comprising:
an inclination sensor;
a pressure sensor;
a wireless transceiver capable of communicating with an external programmer; and
an embedded microprocessor, capable of reading said inclination sensor and said pressure sensor and transmitting, using said wireless transceiver, said readings from said sensors;
and an external programmer with wireless capability, said programmer capable of wireless communication with said controller.

2. The system of claim 1, whereby said programmer can wirelessly transmit data and commands to said implanted controller, and whereby said controller can wirelessly transmit data and status responses to said programmer.

3. A method of determining, in an implanted controller system having a ventricular cannula in fluid communication with the cerebrospinal fluid in the brain of the patient, and a pressure sensor in fluid communication with said ventricular cannula and with an outlet cannula having a distal end, the cerebrospinal fluid flow resistance downstream from said pressure sensor, the method comprising the steps of:
i. Providing a known calibration constant;
ii. measuring with said pressure sensor, a pressure indicative of the initial intraventricular pressure of said patient in a supine position;
iii. occluding the flow of cerebrospinal fluid (CSF) from the brain for a predetermined period;
iv. measuring a plurality of occluded pressures over said predetermined period;
v. storing, said occluded pressure measurements;
vi. determining the difference between said initial pressure and the final pressure of the last of said plurality of occluded pressure measurements; vii. determining the decay time from said initial pressure measurement until one of said occluded pressure measurements reached a value of said initial pressure less half the difference between said initial pressure reading and said final pressure reading; viii. calculating the distal flow resistance, by multiplying said decay time by said calibration constant.

4. The method of claim 3, wherein said controller comprises an occluder located in the CSF flow path between the brain and the pressure sensor and positioned directly beneath the scalp, whereby said occlusion of CSF is achieved by actuating said occluder.

5. The method of claim 3, whereby said occluded pressure measurements are transmitted wirelessly to external programmer before performing steps vi, vii and viii.

6. The method of claim 3, further comprising the step of calculating the supine cerebrospinal fluid flow rate, said flow rate defined as said initial pressure less said final pressure, divided by said distal resistance.

7. The method of claim 3, further comprising the measurement the cranial compliance of a patient with said implanted controller, where said controller comprises a multi mode drainage system in which a first mode is a low resistance passive substantially supine mode and a second mode is a variable pressure substantially upright mode, said measurement comprising the steps of:
i. Sensing an initial upright pressure, where said patient is in an upright position;
ii. changing said drainage mode of the implanted controller to permit the cerebrospinal fluid to flow through said low resistance flow path;
iii. measuring a plurality of upright pressures over a predetermined amount of time;
iv. storing said upright pressure measurements;
v. calculating the instantaneous flow rate corresponding to each said upright pressure measurements, said flow rate being equal to said upright pressure reading less said final pressure reading, divided by said distal flow resistance;
vi. calculating the total volume of CSF shunted by summing all said instantaneous flow rates and multiplying said summation by the sample time, where said sample time is defined as the time between each of said plurality of upright pressure measurements; and
vii. calculating the cranial compliance by dividing said volume by the difference between said initial upright pressure and said final pressure;

8. The method of claim 9, whereby said upright pressure readings are transmitted wirelessly to said external programmer prior to completing steps v, vi, and vii.

9. The method of claim 9, further comprising the step of calculating the proximal shunt resistance between the proximal tip of said ventricular cannula located in the ventricle of said patient and said implanted controller; said proximal shunt resistance given as said decay time, divided by the product of 0.7 and said cranial compliance, then reduced by said distal flow resistance.

10. A method of regularly monitoring cerebrospinal fluid shunt flow resistance in an implanted CSF shunt system, where said shunt system comprises a multi mode drainage system, in which a first mode is a low resistance substantially supine flow path and a second mode is a variable upright mode, which further comprises a check valve with a programmable variable cracking pressure, comprising the steps of:
i. activating the implanted CSF controller at a prescribed time;
ii. monitoring an implanted inclination sensor in said controller to insure that the patient is in a supine position;
iii. measuring the initial pressure recorded by an implanted pressure sensor;
iv. changing said drainage mode of said implanted controller to permit the cerebrospinal fluid to flow through said programmable check valve, said change causing said pressure to increase;
v. monitoring said pressure sensor until the pressure reading exceeds said initial pressure by a predetermined amount;
vi. changing said drainage mode of said implanted controller to permit said cerebrospinal fluid to flow through said low resistance flow path; and
vii. measuring the elapsed time from said change to said low resistance flow path until said pressure sensor measures a pressure reading of said initial pressure plus one half of said amount.

11. The method of claim 10, wherein said prescribed time occurs during said patient's typical sleep period.

12. The method of claim 10, wherein said predetermined amount is in the range of 2-6 mm Hg.

13. The method of claim 10, wherein said elapsed time is stored in said controller's internal memory.

14. The method of claim 13, wherein said controller notifies said patient when said elapsed time changes significantly from said previously stored elapsed time.

15. The method of claim 14, wherein said controller notifies said patient by activating a piezo electric buzzer, located in said controller.

16. The method of claim 13, whereby said stored elapsed times can be transmitted wirelessly to an external controller.
